(19) United States
(12) Patent Application Publication
(10) Pub. No.: US 2005/0069879 A1
(43) Pub. Date: Mar. 31, 2005

(54) Method for high sensitivity detection of cytosine-methylation

(76) Inventor: Kurt Berlin, Stahnsdorf (DE)

Correspondence Address: KRIEGSMAN & KRIEGSMAN, 665 FRANKLIN STREET, FRAMINGHAM, MA, 01702, US

(21) Appl. No.: 10/481,695

(22) PCT Filed: Jun. 20, 2002
(86) PCT No.: PCT/DE02/02264

(30) Foreign Application Priority Data
Jun. 22, 2001 (DE) 101 30 800.0

(51) Int. Cl. C12Q 1/68; C12P 19/34

(52) U.S. Cl. 435/6; 435/91.2

(57) ABSTRACT

A method is described for the detection of cytosine methylation in DNA samples, which permits the analysis of DNA to be investigated in the presence of large quantities of background DNA of the same individual. In the first step, a genomic DNA is chemically treated, preferably with a bisulfite (=disulfite, hydrogen sulfite), in such a way that cytosine is converted into a base that is different in its base pairing behavior in the DNA duplex, while 5-methylcytosine remains unchanged. Then segments of the sample DNA are amplified by means of a polymerase reaction. The amplificates are cleaved selectively by enzymes at those position which have a methylation state in the DNA sample that is not characteristic for the DNA to be investigated further, but which is characteristic for background DNA. The DNA that is not cleaved by enzymes is now amplified in another polymerase reaction, and in this way, the DNA to be investigated is concentrated relative to the background DNA that is present. The amplificate is finally investigated with respect to its sequence properties and the methylation state in the DNA to be investigated in the genomic DNA sample is concluded therefrom.

The present invention concerns a method for the detection of cytosine methylation in DNA samples. The method serves particularly for the detection of the presence or absence of cytosine methylation in the DNA to be investigated in samples of an individual, in which background DNA, which is not to be investigated, of the same individual is present, which [background DNA] is distinguished from the DNA to be investigated only with respect to the methylation state.

The levels of observation that have been well studied in molecular biology according to developments in methods in recent years include the genes themselves, the transcription of these genes into RNA and the translation to proteins therefrom. During the course of development of an individual, which gene is turned on and how the activation and inhibition of certain genes in certain cells and tissues are controlled can be correlated with the extent and nature of the methylation of the genes or of the genome. In this regard, pathogenic states are also expressed by a modified methylation pattern of individual genes or of the genome.

5-Methylcytosine is the most frequent covalently modified base in the DNA of eukaryotic cells. For example, it plays a role in the regulation of transcription, in genetic imprinting and in tumorigenesis. The identification of 5-methylcytosine as a component of genetic information is thus of considerable interest. 5-Methylcytosine positions, however, cannot be identified by sequencing, since 5-methylcytosine has the same base-pairing behavior as cytosine. In addition, in the case of a PCR amplification, the epigenetic information which is borne by the 5-methylcytosines is completely lost.

A relatively new method that in the meantime has become the most widely used method for investigating DNA for 5-methylcytosine is based on the specific reaction of bisulfite with cytosine, which, after subsequent alkaline hydrolysis, is then converted to uracil, which corresponds in its base-pairing behavior to thymidine. In contrast, 5-methylcytosine is not modified under these conditions. Thus, the original DNA is converted so that methylcytosine, which originally cannot be distinguished from cytosine by its hybridization behavior, can now be detected by “standard” molecular biology techniques as the only remaining cytosine, for example, by amplification and hybridization or sequencing. All of these techniques are based on base pairing, which is now fully utilized. The prior art, which concerns sensitivity, is defined by a method that incorporates the DNA to be investigated in an agarose matrix, so that the diffusion and renaturation of the DNA are prevented (bisulfite reacts only on single-stranded DNA) and all precipitation and purification steps are replaced by rapid dialysis (Olek A, Oswald J, Walter J. A modified and improved method for bisulphate* based cytosine methylation analysis. Nucleic Acids Res. 1996 Dec. 15;24(24):5064-6). Individual cells can be investigated by this method, which illustrates the potential of the method. Of course, up until now, only individual regions of up to approximately 3000 base pairs long have been investigated; a global investigation of cells for thousands of possible methylation analyses is not possible. Of course, this method also cannot reliably analyze very small fragments of small quantities of sample. These are lost despite the protection from diffusion through the matrix.
*Sic; bisulphite?—Trans. Note

An overview of other known possibilities for detecting 5-methylcytosines can be derived from the following review article: Rein T, DePamphilis M L, Zorbas H. Identifying 5-methylcytosine and related modifications in DNA genomes. Nucleic Acids Res. 1998 May 15; 26 (10): 2255-64.

The bisulfite technique has been previously applied only in research, with a few exceptions (e.g., Zeschnigk M, Lich C, Buiting K, Dörfler W, Horsthemke B. A single-tube PCR test for the diagnosis of Angelman and Prader-Willi syndrome based an allelic methylation differences at the SNRPN locus. Eur J Hum Genet. 1997 March-April; 5(2):94-8). However, short, specific segments of a known gene have always been amplified after a bisulfite treatment and either completely sequenced (Olek A, Walter J. The pre-implantation ontogeny of the H19 methylation imprint. Nat Genet. 1997 November; 17(3): 275-6) or individual cytosine positions have been detected by a “primer extension reaction” (Gonzalgo M L, Jones P A. Rapid quantitation of methylation differences at specific sites using methylation-sensitive single nucleotide primer extension (Ms-SNuPE). Nucleic step (Xiong Z, Laird P W. COBRA: a sensitive and quantitative DNA methylation assay. Nucleic Acids Res. 1997 Jun. 15; 25(12): 25324). Detection by hybridization has also been described (Olek et al., WO 99-28498).

Urea improves the efficiency of bisulfite treatment prior to sequencing of 5-methylcytosine in genomic DNA (Paulin R, Grigg G W, Davey M W, Piper A A. Urea improves efficiency of bisulphate* mediated sequencing of 5-methylcytosine in genomic DNA. Nucleic Acids Res. 1998 Nov. 1; 26(21): 5009-10).
*Sic; bisulphite?—Trans. Note

Other publications which are concerned with the application of the bisulfite technique for the detection of methylation in the case of individual genes are: Grigg G, Clark S. Sequencing 5-methylcytosine residues in genomic DNA. Bioassays **1994 June; 16(6): 431-6. 431; Zeschnigk M, Schmitz B, Dittrich B, Buiting K, Horsthemke B, Dörfler W. Imprinted segments in the human genome: different DNA methylation patterns in the Prader-Willi/Angelman syndrome region as determined by the genomic sequencing method. Hum Mol Genet. 1997 March; 6 (3):387-95; Feil R, Charlton J, Bird A P, Walter J, Reik W. Methylation analysis on individual chromosomes: improved protocol for bisulphate* genomic sequencing. Nucleic Acids Res. 1994 Feb. 25; 22 (4): 695-6; Martin V, Ribieras S, Song-Wang X, R10 MC, Dante R. Genomic sequencing indicates a correlation between DNA hypomethylation in the 5′ region of the pS2 gene and in its expression in human breast cancer cell lines. Gene. 1995 May 19; 157 (1-2): 261-4; WO97/46705, WO95/15373 and WO97/45560.
Sic; Bioessays?—Trans. Note

Another known method is so-called methylation-sensitive PCR (Herman J G, Graff J R, Myohanen S, Nelkin B D, Baylin S B (1996), Methylation-specific PCR: a novel PCR assay for methylation status of CpG islands. Proc Natl Acad Sci USA. September 3; 93(18): 9821-6). For this method, primers are used, which hybridize either only to a sequence that forms by the bisulfite treatment of a DNA which is unmethylated at the respective position, or, vice versa, primers which bind only to a nucleic acid which forms by the bisulfite treatment of a DNA which is unmethylated* at the respective position. Amplificates can be produced accordingly with these primers, the detection of which in turn supplies indications of the presence of a methylated or unmethylated position in the sample to which the primers bind.
*Sic; methylated?—Trans. Note

A newer method is also the detection of cytosine methylation by means of a Taqman PCR, which has become known as “methyl light” (WO 00/70090). It is possible with this method to detect the methylation state of individual positions or a few positions directly in the course of the PCR, so that a subsequent analysis of the products becomes superfluous.

An overview of the prior art in oligomer array production can be derived also from a special issue of Nature Genetics which appeared in January 1999 (Nature Genetics Supplement, Volume 21, January 1999), the literature cited therein and U.S. Pat. No. 5,994,065 on methods for the production of solid supports for target molecules such as oligonucleotides in the case of reduced nonspecific background signal.

Probes with multiple fluorescent labels are used for scanning an immobilized DNA array. Particularly suitable for fluorescent labels is the simple introduction of Cy3 and Cy5 dyes at the 5′-OH of the respective probe. The fluorescence of the hybridized probes is detected, for example, by means of a confocal microscope. The dyes Cy3 and Cy5, in addition to many others, are commercially available.

Matrix-assisted laser desorptions/ionization mass spectrometry (MALDI-TOF) is a very powerful development for the analysis of biomolecules (Karas M, Hillenkamp F. Laser desorption ionization of proteins with molecular masses exceeding 10,000 daltons. Anal Chem. 1988 Oct. 15; 60 (20): 2299-301). An analyte is embedded in a light-absorbing matrix. The matrix is vaporized by a short laser pulse and the analyte molecule is transported unfragmented into the gaseous phase. The analyte is ionized by collisions with matrix molecules. An applied voltage accelerates the ions in a field-free flight tube. Ions are accelerated to varying degrees based on their different masses. Smaller ions reach the detector sooner than larger ones.

MALDI-TOF spectroscopy is excellently suitable for the analysis of peptides and proteins. The analysis of nucleic acids is somewhat more difficult (Gut, I. G. and Beck, S. (1995), DNA and Matrix Assisted Laser Desorption Ionization Mass Spectrometry. Molecular Biology: Current Innovations and Future Trends 1: 147-157). For nucleic acids, the sensitivity is approximately 100 times poorer than for peptides and decreases overproportionally with increasing fragment size. For nucleid acids, which have a multiply negatively charged backbone, the ionization process via the matrix is essentially less efficient. In MALDI-TOF spectroscopy, the choice of matrix plays an imminently important role. Several very powerful matrices, which produce a very fine crystallization, have been found for the desorption of peptides. In the meantime, several effective matrices have been developed for DNA, but the difference in sensitivity has not been reduced thereby. The difference in sensitivity can be reduced by modifying the DNA chemically in such a way that it resembles a peptide. Phosphorothioate nucleic acids, in which the usual phosphates of the backbone are substituted by thiophosphates, can be converted by simple alkylation chemistry into a charge-neutral DNA (Gut, I. G. and Beck, S. (1995), A procedure for selective DNA alkylation and detection by mass spectrometry. Nucleic Acids Res. 23:1367-1373). The coupling of a “charge tag” to this modified DNA results in an increase in sensitivity to the same amount as is found for peptides. Another advantage of “charge tagging” is the increased stability of the analysis in the presence of impurities, which make the detection of unmodified substrates very difficult. Genomic DNA is obtained from DNA of cells, tissue or other test samples by standard methods. This standard methodology is found in references such as Fritsch and Maniatis, Molecular Cloning: A Laboratory Manual, 1989.

After the invention of PCR, numerous variants became known in the next few years, which refine this technique for the amplification of DNA. In particular, multiplexing of the PCR (multiplex PCR) should be mentioned here, whereby more than 2 specific primers are used and thus a plurality of different, specific amplification[s] can be produced in one reaction vessel.

Particularly interesting also is so-called nested PCR, which is used among other things for the detection of particularly small DNA quantities. This type of PCR consists of two amplifications, one following the other, whereby the primers of the second amplification lie within the first amplificate and are not identical to the primers of the first amplification. In this way, a particular specificity is achieved, since the primers of the second amplification only function if the intended fragment has been produced in the first amplification. In contrast, the propagation of any possible byproducts of the first amplification in the second amplification is excluded as much as possible.

Accordingly, a great many methods for methylation analysis are prior art. The present invention, however, will solve the problem that the current methods cannot, of amplifying in a targeted manner a DNA to be investigated which is found in body fluid or serum, when other DNA segments of homologous sequence of another origin are present at the same time.

This would be particularly advantageous, however, since, for example, free DNA from the most varied sources can be found in serum. Since DNA from different sources in an individual normally does not differ in sequence, but does differ in methylation pattern (if one disregards any viral or bacterial DNA that may be present), there is the need for a method that preferably concentrates the DNA which derives from a fully determined source and thus makes it accessible for precise methylation analysis. This is particularly important for the detection of deviant methylation patterns in tumors, which can be detected, for example, from serum in this way.

The DNA to be investigated as well as the otherwise present nucleic acids, which are named background DNA in the following, are generally amplified to the same extent, since the primers used also cannot distinguish between DNA to be investigated and background DNA. One possibility for differentiating these DNAs results, however, from the different methylation patterns. A current method for this purpose is methylation-sensitive PCR, abbreviated MSP (Herman J G, Graff J R, Myohanen S, Nelkin B D, Baylin S B. (1996), Methylation-specific PCR: a novel PCR assay for methylation status of CpG islands. Proc Natl Acad Sci USA. Septempber 3; 93(18): 9821-6).

This method consists of several sub-steps. First, a bisulfite treatment corresponding to the prior art is carried out, which in turn provides that all cytosine bases are coverted to uracil, while the methylated cytosine bases (5-methylcytosine) remain unchanged. In the next step, primers are now used, which are completely complementary to a methylated DNA converted with bisulfite, but not to a corresponding DNA which was originally present unmethylated. When a PCR is conducted with such a primer, this leads to the circumstance that only the originally methylated DNA is amplified. It is correspondingly possible to use a primer, which in contrast only amplifies the unmethylated DNA. In this manner, if the DNA to be analyzed as well as background DNA are present, the DNA fragments to be investigated will be exclusively and selectively produced as long as they are distinguished from the background DNA with respect to their methylation state in a CpG position.

The prior art is now to infer the methylation state or the presence of a DNA to be investigated from the detection of such a DNA molecule to be investigated, which in turn essentially permits a diagnosis, for example, of a tumor disorder in patients, since it is known that, for example, the serum DNA concentration is increased, in part drastically, in tumor patients. Only the DNA originating from the tumors will then be detected, aside from the background DNA. The DNA analysis in other body fluids is essentially comparable.

The prior art is again a method developed by Epigenomics, which amplifies DNA to be investigated and background DNA to the same extent after bisulfite treatment and then the former CpG positions that are contained in the fragment are investigated by hybridization techniques, [or] alternatively by means of minisequencing or other current methods. This has the advantage that one obtains a quantitative pattern with respect to the investigated methylation positions, i.e., it produces a determination of the degree of methylation for a plurality of positions, which makes possible a very precise classification, e.g., in the case of solid tumors. The disadvantage of this method, however, is that it cannot supply accurate information in cases in which the background DNA is excessive, since this DNA is amplified precisely along with the DNA to be investigated and both are analyzed in the mixture. This problem does not exist in the analysis of solid tumors, where one can select the material to be investigated in a targeted manner, but it can complicate the analysis of serum DNA, for example.

The object of the present invention is now to overcome the disadvantages of the prior art and to combine the advantages of both methods [described above] for the detection of methylation patterns in body fluids and serum. As mentioned above, this is particularly important for the detection of deviant methylation patterns in tumors, which can be detected, for example, from serum in this way. This object is solved by creating a method for the detection of cytosine methylation in DNA samples, in which the following steps are conducted:
a genomic DNA sample which comprises DNA to be investigated and background DNA is chemically treated, preferably with a bisulfite (=hydrogen sulfite, disulfite), such that all unmethylated cytosine bases are converted to uracil, while the 5-methylcytosine bases remain unchanged;
then the chemically treated DNA sample is amplified with the use of preferably at least 2 primer oligonucleotides by means of a polymerase reaction;
the DNA is cut selectively by enzymes at those position which have a methylation state in the DNA sample, which is not characteristic for the DNA to be investigated further, but which is characteristic for background DNA;
the DNA that is not cleaved by enzymes is amplified in another polymerase reaction, by which means the DNA to be investigated is concentrated relative to the background DNA that may be present;
the amplificate is investigated with respect to its sequencing properties and the methylation state in the DNA to be investigated in the genomic DNA sample is concluded therefrom.

It is preferred according to the invention that the DNA samples are obtained from serum or other body fluids of an individual.

It is additionally preferred according to the invention, that the DNA samples are obtained from cell lines, blood, sputum, stool, urine, serum, cerebrospinal fluid, tissue embedded in paraffin, for example, tissue from intestine, kidney, brain, heart, prostate, lung, eyes, breast or liver, histological slides and all possible combinations thereof.

It is most particularly preferred according to the invention that the chemical treatment is conducted with a bisulfite (=disulfite, hydrogen sulfite). It is also preferred that the chemical treatment is conducted after embedding the DNA in agarose. It is also and additionally preferred that in the chemical treatment, a reagent that denatures the DNA duplex and/or a radical trap is present.

It is also particularly preferred to conduct the amplifications of several different fragments with more than 2 different primers in one reaction vessel and thus to carry out the amplification steps as a multiplex PCR. It is generally particularly preferred to conduct the amplifications as a polymerase chain reaction.

The primers used in the amplifications do not most preferably amplify fragments of genomic DNA that is not treated with bisulfite (or only do so to a negligibly small extent), so that they are specific for the DNA converted with bisulfite. This protects from erroneous results in the case of an incomplete conversion reaction with sodium bisulfite, for example.

The enzymatic cleavage of the background DNA is particularly preferably conducted with a restriction endonuclease or several different restriction enzymes. If several restriction endonucleases are used, then it depends on the respective buffers whether these enzymes are applied sequentially or simultaneously. The use of restriction enzymes according to the protocols supplied by the manufacturers is known to the person skilled in the art. The preferred restriction enzymes which are listed below and, which can be commercially obtained, make no claim to completeness: Mae II, Psp 1406 I, Ast II, Ssp 5230 I, Bbr P I, Bsa AI, Sna B I, Cfo I, Hin P1 I, Eco 47 III, NAR I, Ehe I, Kas I, Bbe I, Hae II, Acy I, Ban I, Hgi CI, Aos I, Avi II, Hpa II, Msp I, Pin AI, Age I, Eco 56 I, Nae I, Cfr10I, SgrAI, Fse I, XmaCI, Sma I, Srf I, Ava I, Bse AI, Mro I, Taq I, CIa I, Sal I, Hind III, Acc I, Xho I, Sfu I, BstBI, Hinf I, Sau 96 I, Dra II, PssI, Ita I, Dsa V, Scr F I, Mae III, Bst E II, Dde I, Cel II, Esp I or Aoc I.

Since they must distinguish between TG and CG dinucleotides, or between CG and CA on the counterstrand, after the bisulfite conversion, the restriction enzymes cleave all of the sequences that contain one of these motifs.

Consequently, the restriction endonucleases most preferably cleave either at positions which corresponded to an essentially methylated CpG position in the DNA to be investigated prior to the bisulfite conversion and amplification, while the background DNA at this position was present essentially unmethylated, and/or the restriction endonucleases cleave at positions which corresponded to an essentially unmethylated CpG position in the DNA to be investigated prior to the bisulfite conversion and amplification, while the background DNA at this position was present essentially methylated.

In a particularly preferred variant of the method, at least 90% of all fragments produced in the previous amplification are cleaved in the restriction step. This is particularly the case for the appropriate completeness of this enzymatic step, when the DNA to be investigated makes up less than 10% of the total DNA. This [small content] is particularly preferred, however, since then the advantages of the method presented here are particularly apparent when compared to conventional techniques: [namely,] the high specificity based on the two different amplifications and the high selectivity for the DNA to be investigated.

Therefore, in the second amplification step, additional or exclusive primers are particularly preferably used, which hybridize to the amplificates of the first step, but not with the primers of the first amplification step or not to segments with substantially homologous sequence. Thus, in this method, a nested PCR is conducted, wherein the fragments that are associated with the background DNA are enzymatically cleaved between the amplification steps. These fragments can then no longer serve as templates for a PCR in the following amplification, and consequently, the DNA to be investigated is amplified exclusively. Since this is the case, it is particularly preferred if the restriction cleavage sites lie within the sequence segment that is also to be amplified in the second amplification. Nevertheless, a variant of the method is also preferred, in which the same set of primers is used in both amplification steps. This [variant] is then particularly advantageous, if high-degree multiplexed PCR is conducted, since establishing these reactions is time-consuming, and in this way, one is spared twice preparing the set of primers belonging thereto with the appropriate reaction conditions. This is done at the expense of the specificity of these amplifications.

It is particularly preferred that the first amplification step is conducted as a multiplex PCR. Conducting both amplification steps as a multiplex PCR is also particularly preferred.

A variant is also particularly preferred, in which the primers of the second amplification step overlap with the cleavage sites of the restriction endonuclease(s). In this case, the hybridization of the primers to the cleaved amplificates is prevented a priori in this step, which again promotes the specific amplification of the fragments deriving from the DNA to be investigated.

It is further preferred according to the invention that the background DNA is present in 100× the concentration in comparison to the DNA to be investigated. It is further preferred that the background DNA is present in 1000× the concentration in comparison to the DNA to be investigated.

It is further preferred that the analysis or the additional analysis is optionally conducted by means of hybridization to oligomer arrays, wherein the oligomers can be nucleic acids or molecules such as PNAs that are similar in their hybridization properties.

It is also advantageous according to the invention that the oligomers hybridize to the DNA to be analyzed over a 12-22 base long segment and that they comprise a CG, TG or CA dinucleotide.

It is preferred that the methylation state of more than 10 methylation positions of the DNA to be analyzed is detected in one experiment.

It is additionally preferred that the methylation state of more than 60 methylation positions of the DNA to be analyzed is detected in one experiment.

It is also particularly preferred according to the invention that the analysis or optionally the further analysis is conducted by measuring the length of the amplified DNA to be investigated, whereby methods for length measurement comprise gel electrophoresis, capillary gel electrophoresis, chromatography (e.g. HPLC), mass spectrometry and other suitable methods. It is also advantageous that methods for sequencing comprise the Sanger method, the Maxam-Gilbert method, and other methods such as sequencing by hybridization (SBH).

A method is also preferred according to the invention, wherein the sequencing is carried out for each CpG position or a small group of CpG positions, each with a separate primer oligonucleotide and the extension of the primer makes up only one or just a few bases and the methylation state of the respective positions in the DNA to be investigated is concluded from the type of primer extension.

It is again preferred that a conclusion is made on the presence of a disease or another medical condition of the patient from the methylation degree of the different CpG positions investigated.

It is advantageous that the amplificates themselves are provided with a detectable label for the detection. These labels are preferably introduced in the generated fragments either by a labeling of the primers or the nucleotides during the amplification.

It is again advantageous that the labels are fluorescent labels or/and that the labels are radionuclides or/and that the labels are removable mass labels, which are detected in a mass spectrometer.

It is further preferred that in the amplification, one of the primers is bound to a solid phase. For example, this solid phase can involve functionalized polymers, metals, glass or semiconductors such as silicon. The primers are linked preferably via bifunctional linker molecules, which are bound to a silanized surface or, for example, via thioates in the primer to bromoacetyl derivatized surfaces or gold.

It is also [preferred] according to the invention that all the amplificates are detected in the mass spectrometer and are thus clearly characterized by their mass.

It is also particularly preferred to observe the formation of specific fragments during the amplification with the use of reporter oligonucleotides, which change their fluorescent properties by specific interaction with the respective amplificate and other oligonucleotides, primers and/or the polymerase.

It is therefore advantageous that in addition to the reporter oligonucleotide, another oligomer which is labeled with a fluorescent dye is used, which hybridizes right next to the reporter oligonucleotide and this hybridization can be detected by means of fluorescence resonance energy transfer. In addition, it is advantageous that a Taqman assay is conducted. It is also preferred that a LightCycler assay is conducted. In addition, it is preferred that the reporter oligonucleotides bears at least one fluorescent label. It is also preferred that the reporter molecules indicate the amplification either by an increase or a decrease in the fluorescence. It is particularly advantageous that the increase or the decrease in the fluorescence is also used directly for the analysis and a conclusion on the methylation state of the DNA to be analyzed is made from the fluorescent signal.

Another subject of the present invention is also the use of a method according to the invention for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as [a consequence of] an abnormality in the development process; malfunction, damage or disorder of the skin, the muscles, the connective tissue or the bones; endocrine and metabolic malfunction, damage or disease; headaches or sexual malfunction.

The use of a method according to the invention is thus advantageous for distinguishing cell types or tissues or for investigating cell differentiation.

The subject of the present invention is also a kit consisting of a reagent containing bisulfite, primers for producing the amplificates, as well as, optionally, instructions for conducting an assay according to the invention.

The present invention thus describes a method for the detection of the methylation state of genomic DNA samples. In contrast to the methods which were known previously, the methylation degree of a set of CpG positions is determined in a selected subgroup of DNA fragments, e.g., in serum, so that an analysis is also possible in the presence of an excess of diagnostically irrelevant background DNA.

The fragments obtained in the second amplification step are analyzed based on their methylation signature, and the degree of methylation of preferably several former CpG positions is determined in the amplificates. Preferably a conclusion is made on the presence of a disease or another medical condition of the patient from the methylation degree of the different CpG positions investigated.

The essence of the present invention is now that two types of CpG positions play a role and contribute equally to the analysis and these will be called below “qualifier” positions and “classifier” positions. The qualifier positions serve for the purpose of distinguishing between the two amplification steps, in the case of enzymatic cleavage, between the DNA to be analyzed and the background DNA. This [distinguishing] can be carried out technically in different ways. The [particular] property of these positions is, however, that their degree of methylation in the DNA to be investigated differs as much as possible from that in the background DNA. The classifier positions, in contrast, serve for the purpose of extracting information on the respective degree of methylation, which is important for the diagnosis, from the amplificate which is produced predominantly from the DNA to be investigated. Up to several hundred of such classifier positions can be used for an analysis, and the analysis is produced, for example, on oligomer arrays, although this is often not necessary. In this case, however, the formation of a specific amplificate is of lesser importance for the results of investigation than is the analysis of the CpG positions in the same amplificate. This basically distinguishes the method described here from other known methods such as MSP, which are used for methylation analysis. In several cases, however, it is certainly possible and meaningful to include information in the analysis, which is derived from the formation of an amplificate, so in this case several positions are then both classifier and qualifier.

One possible procedure that is particularly preferred is described in the following.

The first step of the method, the obtaining of samples, is preferably conducted by sampling of body fluids, such as, e.g., sputum or serum, but it is obvious that the method can be conducted with many different kinds of samples from different sources.

The DNA is purified or concentrated in several cases prior to the bisulfite treatment in order to avoid a disruption of the bisulfite reaction and/or the subsequent PCR by too high a content of impurities. However, it is known that, for example, a PCR can be conducted from tissue, after treatment for example, with proteinase K without further purification, and this logically follows also for the bisulfite treatment and subsequent PCR.

The chemical treatment is preferably conducted by treatment with a bisulfite (=hydrogen sulfite, disulfite), more preferably sodium bisulfite (ammonium bisulfite is less suitable). The reaction is either conducted according to a published variant, and preferably the DNA here is embedded in agarose, in order to keep the DNA in the single-stranded state during treatment, or, however, according to a new variant, by treatment in the presence of a radical trap and a denaturing reagent, preferably an oligoethylene glycol dialkyl ether or, for example, dioxane. Prior to the PCR reaction, the reagents are removed either by washing in the case of the agarose method or a DNA purification method (prior art, precipitation or binding to a solid phase, membrane) or, however, are brought simply by dilution to a concentration range which no longer significantly influences the PCR.

It is now essential for the next step that the qualifier positions are selected and a suitable restriction enzyme is selected, which permits selective cleavage of the background DNA not to be analyzed. The positions are thus selected according to the premise that they should distinguish as much as possible between the methylation [state] of the background DNA and that of the DNA to be investigated, and also a suitable enzyme must be available for the corresponding sequence context. The restriction enzyme must also be selected so that cleavages are not produced in other desired amplificates.

First, the methylation profiles are determined for the segments of a gene that are in question each time, both for tumors to be investigated as well as for the background DNA of healthy individuals. Those positions, which have the greatest differences between tumor DNA and background DNA (for example in serum) will be selected as qualifier positions. Such positions are already known for a plurality of genes, for example, for GSTpi, for HIC-1 and MGMT (von Wronski M A, Harris L C, Tano K, Mitra S, Bigner D D, Brent T P. (1992) Cytosine methylation and suppression of O6-methylguanine DNA methyltransferase expression in human rhabdomyosarcoma cell lines and xenografts. Oncol Res.; 4 (4-5): 167-74; Esteller M, Toyota M, Sanchez-Cespedes M, Capella G, Peinado M A, Watkins D N, Issa J P, Sidransky D, Baylin S B, Herman J G. (2000), Inactivation of the DNA repair gene O6-methylguanine-DNA methyltransferase by promoter hypermethylation is associated with G to A mutations in K-ras in colorectal tumorigenesis. Cancer Res. May 1; 60 (9): 2368-71).

The chemically treated DNA is amplified in principle, as it is prior art, by means of at least two primers; a multiplexing is possible. One or more qualifier positions, and preferably also one or more classifier positions are found within the DNA segment bounded by the two primers. The sequence of these positions is not important for setting up the assay described here.

As presented above, a digestion with one or more restriction enzymes, depending on the number of qualifier positions each time, is conducted after the first amplification. After the digestion, the primers, enzymes and possibly nucleotides that may still be present, are removed either by a purification step (for example, an ethanol precipitation can be conducted, or a common commercial purification kit for PCR products is used) or the solution is diluted so much with PCR buffer for the second amplification that the above-named components do not matter in this reaction.

As discussed above, several qualifier positions and also, accordingly, several restriction enzymes that are each specific for a methylation state present in the background DNA and thus specific for a certain sequence after bisulfite conversion are used.

The primers of the second amplification preferably lie within the fragment produced in the first amplification and in such a way that they neither significantly overlap with the previously used primers nor hybridize with them. A nested PCR is thus conducted. Care is to be taken that the qualifier positions lie within the second amplificate so that the method functions properly. An overlapping of the primers of the second amplification with the qualifier position is possible. If the latter is found too near the 3′ end of the primer, in the case of cleaved DNA, an amplification can no longer occur.

After the selective, second amplification of the DNA to be investigated, the methylation state of several classifier positions can now preferably be determined according to methods, which are known in and of themselves.

It is obvious that even in this case, the emergence of a PCR fragment itself can provide sufficient information in the individual case, since the situation is thus present, as it is also in MSP, that the qualifier position is unmethylated practically up to 100%, for example, in the background DNA, but is methylated in the DNA to be investigated. If one now uses in the PCR an oligonucleotide, which preferably binds to the sequence which forms in the bisulfite treatment from unmethylated background DNA, then only one product is then formed in the PCR, when at least a small quantity of the DNA to be investigated is present overall. This may even be sufficient for a diagnosis in the individual case, and it would involve a method that has an application potential similar to MSP. Although such a procedure is not directly preferred, such a method has not previously been known and is consequently also considered to belong to the subject of this invention.

It is preferred that in a PCR reaction, several fragments are generated simultaneously, i.e., that a multiplex PCR is conducted. In the case of bisulfite-treated DNA, one thus has the advantage that a forward primer can never function also as a reverse primer, due to the different G and C content of the two DNA strands, which facilitates the multiplexing.

In the simplest case, the fragments that are formed are now detected without obtaining individual information on the degree of methylation of the CpG positions previously present in them. For this purpose, all possible known molecular biology methods are considered, such as gel electrophoresis, sequencing, liquid chromatography or hybridizations, without separately analyzing the classifier positions. The same considerations are also conceivable for the quality control of the preceding method steps. As indicated above, however, the subsequent analysis of the degree of methylation of classifier positions is particularly preferred.

There are numerous possibilities for combining the preferred amplification of the DNA to be investigated in the second amplification step advantageously with detection techniques for the classifier oligonucleotides.

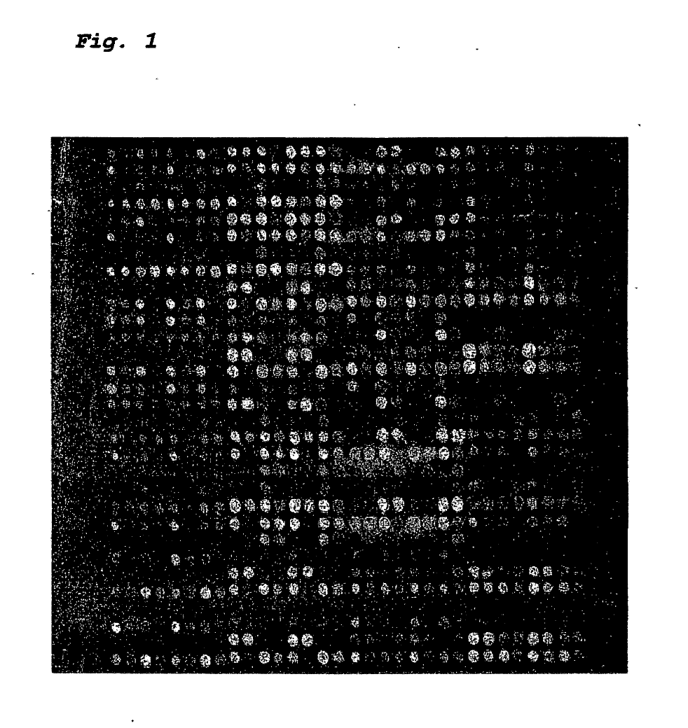

Detection techniques, which are particularly suitable for this, are hybridization to oligomer arrays and, for example, primer extension (minisequencing) reactions. Hybridization to oligomer arrays can be used without further change of protocols when compared with the closest prior art (Olek A, Olek S, Walter J; WO-Patent 99-28498). It is preferred, however, to hybridize the amplificates to an array of oligomers, which consists of pairs of oligonucleotides immobilized to a solid phase, one of which hybridizes most preferably to a DNA segment containing an originally unmethylated CpG (classifier position) and the other in turn hybridizes most preferably to the corresponding segment in which originally a methylated CpG was contained, each time prior to the bisulfite treatment and amplification. In this case, the amplificate or the amplificates are particularly preferably labeled fluorescently or radioactively or with removable mass tags, so that after the hybridization, the fragments bound to both oligonucleotides of a pair can be detected and quantified on the basis of this label. An intensity ratio is obtained from which, for example, the degree of methylation of the respective classifier position can be determined after calibration of the experiment with completely methylated and completely unmethylated DNA. A plurality of fragments and classifier positions can be detected simultaneously on such an oligomer array (FIG. 1).

It is meaningful and preferred that the array also contains oligomers detecting qualifier positions for the control of the experiment, since, the ratio of the DNA to be investigated, which enters into the analysis, to the background DNA, can be determined.

Primer extension reactions can also be conducted on oligonucleotides immobilized on a solid phase. Although not absolutely necessary, the immobilizing of these primers is preferred, since usually a plurality of classifier positions from several amplificates will be investigated and this can be conducted on a solid phase, thus on an oligomer array, significantly more easily and in one experiment. It is particularly preferred that the primers are found directly next to a classifier position and that the extension occurs only by one nucleotide. It is particularly preferred that only dideoxythymidine and dideoxycytidine are added as nucleotides and that these are each labeled with a different fluorescent dye, whereby, of course, other distinguishable labels such as mass tags are also conceivable and preferred. After a bisulfite treatment and amplification, previously methylated CGs are present as CGs and unmethylated CGs are now present as TGs. The primer extension reaction thus leads to the incorporation of a dideoxycytidine or a dideoxythymidine. The degree of methylation of the respective position can be concluded from the ratio of the fluorescent labels detected each time for these two terminators. It is also possible and preferred in this case, to conduct the primer extension with deoxycytidine and deoxythymidine, if one does not add a guanine derivative, and consequently for a TG or CG sequence, the primer extension terminates even after one base, without anything further. In addition, it is also preferred to conduct the analysis analogously on the counterstrand by distinguishing CA and CG, then correspondingly proceeding with dideoxy-ATP und dideoxy-GTP or their derivatives.

A particularly preferred variant of the method is, however, the simultaneous detection of qualifier positions and classifier positions in one experiment, which can be achieved by use of Taqman or LightCycler technology variants (real time PCR). In this special case of the present method, the second amplification is conducted as real time PCR with corresponding reporter oligonucleotides, which can bind to different classifier positions. Thus, pairs of such reporter oligonucleotides are preferably used, wherein one of the oligonucleotides preferably binds to the sequence which corresponds to a methylated position prior to the bisulfite treatment and the other of which binds to the sequence forming from a corresponding unmethylated position. In this way, additional fluorescently labeled oligonucleotides are added to the oligonucleotides which are provided for one amplification of the DNA to be investigated, and the change in fluorescence during the PCR reaction is measured. Since the DNA to be investigated is amplified, information on the methylation state of different classifier CpG positions is obtained for the most part also directly from this change in fluorescence.

Since different oligonucleotides are each preferably provided with a different fluorescent dye, a differentiation of the change in fluorescence during the PCR is also possible separately for different positions.

This change in fluorescence dependent on the methylation state can be obtained by numerous methods, two of which will be introduced here by way of example.

First of all, oligonucleotide probes can be used, which bind specifically either to a sequence which is produced by chemical treatment of an unmethylated DNA at the corresponding position, or to a sequence which is produced by chemical treatment of a methylated DNA at the corresponding position. These probes are particularly preferably provided with two fluorescent dyes, a quencher dye and a fluorescent dye serving as a marker. Both are coupled to these oligonucleotide probes. Now if a PCR reaction occurs with the DNA to be investigated as the template, then the PCR reaction is blocked this time by the fluorescently-labeled oligomer probes. However, since this is not resistent to the nuclease activity of the polymerase, a decomposition of the probe bound to the template DNA occurs during the PCR reaction, which correlates with the binding efficiency of the probe to the template, since the unbound probe is not decomposed by the polymerase. The decomposition of the probe is now directly visible by an increase of the fluorescence of the marker dye, because the quencher dye and the fluorescent dye serving as the marker are separated from one another. In principle, this involves a variant of the so-called Taqman assay.

Accordingly, what is measured is the formation of a PCR product from the DNA to be investigated, but only when the investigated classifier position is also present in the methylation state that the probe can detect by hybridization to the chemically treated DNA. A cross-check with a probe that would bind correspondingly to the classifier position in the other methylation state, is thus appropriate and preferred. It can be operated in principle also only with one probe, which once more need not absolutely bind to a classifier position, and only the presence of a specific methylation state in the qualifier position can be concluded directly from the formation of the PCR product.

Different fluorescent dyes with different emission wavelengths for several probes are preferably utilized together with the quencher, in order to be able to distinguish among the probes and thus to achieve a multiplexing.

It is also preferred that several positions can be simultaneously investigated for their degree of methylation with one probe.

If a more precise quantification of the degree of methylation of the classifier positions is desired, then two probes competing with one another and having different dyes can also be utilized preferably, whereby one of these again preferably binds in the case of an unmethylated position in the DNA to be investigated, while the other preferably binds in the case of a methylated position The methylation state of the investigated position can then again be concluded from the ratio of the increases in fluorescence for the two dyes.

A basically different method, in which, however, there is also a change in fluorescence during the PCR, is known presently as LightCycler™ technology. The fact is utilized that a fluorescence resonance energy transfer (FRET) can only occur between two dyes, if these are spaced in the immediate vicinity to one another, i.e., within 1-5 nucleotides. Only then can the second dye be excited by the emission of the first dye, and then in its turn, emit light of another wavelength, which is then detected.

In the present case of methylation analysis, a hybridization of a fluorescently labeled probe to the respective chemically treated DNA occurs at a classifer position, and the binding of this probe depends in turn on whether the DNA to be investigated was methylated or unmethylated at this position. Another probe with another fluorescent dye binds directly adjacent to this probe. This binding preferably occurs in turn as a function of sequence and thus of methylation, if another methylatable position is present in the respective sequence segment (always after bisulfite treatment). During the amplification, the DNA is now amplified, for which reason continuously more fluorescently labeled probes bind adjacent to the position in question, insofar as these had the methylation state necessary for this prior to the bisulfite treatment, and thus an increasing FRET is measured.

A multiplexing with several different fluorescently labeled probes is also produced preferably by this method.

The two methods differ in result principally in that in one case a decrease in fluorescence is measured, whereas an increase is measured in the other case. Qualifier as well as classifier positions can be measured in both cases.

The following examples explain the invention:

### EXAMPLE 1

Conducting the Method on the Example of the ELK-1 Gene

The DNA isolated from serum with the use of bisulfite (hydrogen sulfite, disulfite) is treated in such a way that all of the unmethylated cytosines at the 5-position of the base are converted to uracil, while the cytosines that are methylated in the 5-position remain unchanged. The agarose method, which is known in the prior art and is described above, is used for this reaction. The first amplification of a defined fragment of 530 bp in length from the promoter region of the ELK-1 gene is now conducted by means of two primer oligonucleotides ATGGTTTTGTTTAATYGTAGAGTTGTTT (SEQ-ID: 1) and TAAACCCRAAAAAAAAAAACCCAATAT (SEQ-ID: 2). In order to remove all amplificates methylated in the 29th former genomic CpG position of this fragment, the amplificate is first isolated by ethanol precipitation and then incubated with Mae II according to the data of the manufacturer (Roche Molecular Biochemicals). After digestion has been conducted, the solution is diluted 1:10,000 with PCR buffer and a second amplification is conducted with the primer oligonucleotides TTTATTTTTATATAAGTTTTGTTT (SEQ-ID: 3) and CCCTTCCCTACAAAACTATAC (SEQ-ID: 4). These primer oligonucleotides are labeled with the fluorescent dye Cy5, and thus the fragment obtained in the PCR is also labeled.

### EXAMPLE 2

Conducting the Hybridization and Evaluating a Hybridized DNA Chip

The amplificate prepared in Example 1 is hybridized to a DNA chip. Oligonucleotides have been previously immobilized on the chip. The oligonucleotide sequences are derived from the amplified fragment of the ELK-1 gene named in Example 1, and represent the CG dinucleotides, including their immediate surroundings. The length of the oligonucleotides amounts to 14-22 nucleotides; the position of the CG dinucleotide within the oligonucleotide is variable. After the hybridization (5 h, 38° C., 5×SSC), the DNA chip is measured on a fluorescence scanner (Genepix 4000A) and the hybridization signals are numerically evaluated.

The appended figures also serve for explaining the invention.

A DNA chip is shown in FIG. 1 after hybridization with the promoter fragment. The pseudo-color image as it is produced after scanning is shown. Unlike the black-and-white illustration shown here, a color image is produced by the scanner. The intensity of the different colors represents the degree of hybridization, whereby the degree of hybridization decreases from red (this can be recognized as light spots in FIG. 1) to blue (recognized as dark spots in FIG. 1).

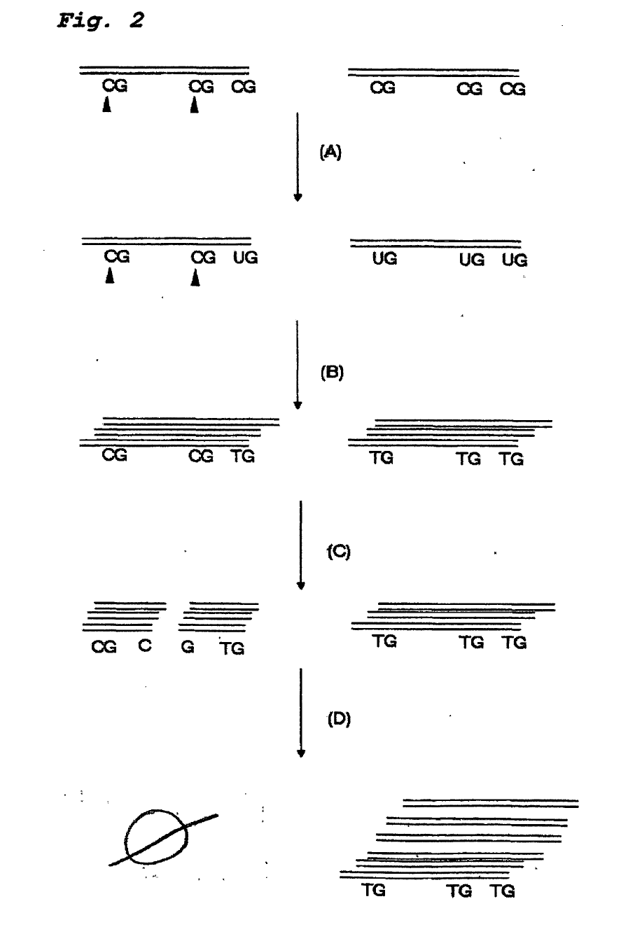

FIG. 2 shows schematically the basic procedure of the method according to the invention. The background DNA, which is not to be analyzed and which in this example is present partially methylated, is shown on the left side, while the unmethylated DNA to be analyzed is shown correspondingly on the right side. In the first step (A), a bisulfite conversion occurs, while in the second step (B) the first amplification takes place. In the third step (C), the background DNA, which is not to be analyzed, is cleaved and the remaining DNA is then again amplified (D). Its sequence properties then permit conclusions on the methylation state of the DNA to be investigated at essentially all positions which are found in the amplified region.

1. A method for the detection of cytosine methylation in DNA samples is hereby characterized in that the following method steps are conducted:
a genomic DNA is chemically treated, preferably with a bisulfite (=disulfite, hydrogen sulfite), in such a way that cytosine is converted into a base that is different in its base pairing behavior in the DNA duplex, while 5-methylcytosine remains unchanged,
segments of the sample DNA are amplified by means of a polymerase reaction,
the DNA is cleaved selectively by enzymes at those position which have a methylation state in the DNA sample, which is not characteristic for the DNA to be investigated further, but which is characteristic for background DNA,
the DNA that is not cleaved by enzymes is amplified in another polymerase reaction, by which means the DNA to be investigated is concentrated relative to the background DNA that is present,
the amplificate is investigated with respect to its sequence and the methylation state in the DNA to be investigated in the genomic DNA sample is concluded therefrom.

2. The method according to claim 1, further characterized in that the DNA samples are obtained from serum or other body fluids of an individual.

3. The method according to claim 1, further characterized in that the DNA samples are obtained from cell lines, blood, sputum, stool, urine, serum, cerebrospinal fluid, tissue embedded in paraffin, for example, tissue from eyes, intestine, kidney, brain, heart, prostate, lung, breast or liver, histological slides and all possible combinations thereof.

4. The method according to claim 1, further characterized in that the chemical treatment is conducted with a bisulfite (=disulfite, hydrogen sulfite).

5. The method according to claim 4, further characterized in that the chemical treatment is conducted after embedding the DNA in agarose.

6. The method according to claim 4, further characterized in that, in the chemical treatment, a reagent that denatures the DNA duplex and/or a radical trap is present.

7. The method according to claim 1, further characterized in that the amplification of several fragments is conducted in one reaction vessel in the form of a multiplex PCR.

8. The method according to claim 1, further characterized in that the primers utilized in the amplification amplify the DNA which has been chemically converted with bisulfite, but not the corresponding unconverted genomic sequence.

9. The method according to claim 1, further characterized in that the enzymatic cleavage is produced by means of a restriction endonuclease.

10. The method according to claim 9, further characterized in that the restriction endonucleases include Mae II, Psp 1406 I, Ast II, Ssp 5230 I, Bbr P I, Bsa AI, Sna B I, Cfo I, Hin P1 I, Eco 47 III, NAR I, Ehe I, Kas I, Bbe I, Hae II, Acy I, Ban I, Hgi CI, Aos I, Avi II, Hpa II, Msp I, Pin AI, Age I, Eco 56 I, Nae I, Cfr10I, SgrAI, Fse I, XmaCI, Sma I, Srf I, Ava I, Bse AI, Mro I, Taq I, Cla I, Sal I, Hind III, Acc I, Xho I, Sfu I, BstBI, Hinf I, Sau 96 I, Dra II, PssI, Ita I, Dsa V, Scr F I, Mae III, Bst E II, Dde I, Cel II, Esp I or Aoc I.

11. The method according to claim 9, further characterized in that several restriction endonucleases are applied.

12. The method according to claim 11, further characterized in that several different restriction endonucleases are applied in one reaction vessel.

13. The method according to claim 1, further characterized in that, in the restriction step, at least 90% of all fragments produced in the previous amplification are cleaved.

14. The method according to claim 1, further characterized in that the same primers are used in the second amplification step as in the first amplification step.

15. The method according to claim 1, further characterized in that, in the second amplification step, additional or exclusive primers are used, which hybridize to the amplificates of the first step, but are essentially not identical to the primers of the first step or hybridize with them (nested PCR).

16. The method according to claim 1, further characterized in that the second amplification step is conducted as a multiplex PCR.

17. The method according to claim 1, further characterized in that primers of the second amplification step overlap with the cleavage sites of the restriction endonuclease(s) utilized in the preceding step.

18. The method according to claim 1, further characterized in that the background DNA is present in 100× the concentration in comparison to the DNA to be investigated.

19. The method according to claim 1, further characterized in that the background DNA is present in 1000× the concentration in comparison to the DNA to be investigated.

20. The method according to claim 1, further characterized in that the analysis of the sequence properties of the amplificates is made by means of hybridization to oligomer arrays, whereby the oligomers can be nucleic acids or molecules such as PNAs that are similar in their hybridization properties.

21. The method according to claim 20, further characterized in that the oligomers hybridize to the DNA to be analyzed over a 12-22 base long segment and comprise a CG, TG or CA dinucleotide.

22. The method according to one of claims 20 or 21, further characterized in that the methylation state is detected for more than 10 methylation positions of the DNA to be analyzed in one experiment.

23. The method according to one of claims 20 or 21, further characterized in that the methylation state is detected for more than 60 methylation positions of the DNA to be analyzed in one experiment.

24. The method according to claim 1, further characterized in that the analysis is conducted by measuring the length of the amplified DNA to be investigated, whereby methods for length measurement comprise gel electrophoresis, capillary gel electrophoresis, chromatography (e.g. HPLC), mass spectrometry and other suitable methods.

25. The method according to claim 1, further characterized in that the analysis is conducted by sequencing, whereby methods for sequencing comprise the Sanger method, the Maxam-Gilbert method, and other methods such as sequencing by hybridization (SBH).

26. The method according to claim 25, further characterized in that the sequencing is carried out for each CpG position or a small group of CpG positions, each with a separate primer oligonucleotide and the extension of the primer makes up only one or just a few bases and the methylation state of the respective positions in the DNA to be investigated is concluded from the type of primer extension.

27. The method according to claim 1, further characterized in that a conclusion is made on the presence of a disease or another medical condition of the patient from the methylation degree of the different CpG positions investigated.

28. The method according to claim 1, further characterized in that the amplificates themselves are provided with at least one detectable label for the detection, which label is introduced either by labeling of the primers or the nucleotides during the amplification.

29. The method according to claim 28, further characterized in that the labels are fluorescent labels.

30. The method according to claim 28, further characterized in that the labels are radionuclides.

31. The method according to claim 28, further characterized in that the labels are removable mass labels which are detected in a mass spectrometer.

32. The method according to claim 1, further characterized in that, in at least one of the amplifications, one of the respective primers is bound to a solid phase.

33. The method according to 1, further characterized in that all of the amplificates are detected in the mass spectrometer and are thus clearly characterized by their mass.

34. The method according to claim 1, further characterized in that the formation of specific fragments during the amplification is observed with the use of reporter oligonucleotides, which change their fluorescent properties by interaction with the amplificate and/or the polymerase.

35. The method according to claim 34, further characterized in that, in addition to the reporter oligonucleotide, another oligomer which is labeled with a fluorescent dye is used, which hybridizes to the amplificate right next to the reporter oligonucleotide and this hybridization can be detected by means of fluorescence resonance energy transfer.

36. The method according to one of claims 34 or 35, further characterized in that a Taqman assay is conducted.

37. The method according to one of claims 34 or 35, further characterized in that a LightCycler assay is conducted.

38. The method according to claim 34, further characterized in that the reporter oligonucleotides bear at least one fluorescent label.

39. The method according to claim 34, further characterized in that the reporter molecules indicate the amplification either by an increase or a decrease of the fluorescence.

40. The method according to claim 39, further characterized in that the increase or the decrease in the fluorescence is used directly for the analysis and a conclusion on the methylation state of the DNA to be analyzed is made from the fluorescent signal.

41. Use of a method according to claim 1 for the diagnosis and/or prognosis of adverse events for patients or individuals, whereby these adverse events belong to at least one of the following categories: undesired drug interactions; cancer diseases; CNS malfunctions, damage or disease; symptoms of aggression or behavioral disturbances; clinical, psychological and social consequences of brain damage; psychotic disturbances and personality disorders; dementia and/or associated syndromes; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as a consequence of an abnormality in the development process; malfunction, damage or disorder of the skin, the muscles, the connective tissue or the bones; endocrine and metabolic malfunction, damage or disease; headaches or sexual malfunction.

42. Use of a method according to claim 1 for the differentiation of cell types or tissues or for the investigation of cell differentiation.

43. A kit, consisting of a reagent containing bisulfite, primers for the production of amplificates, as well as, optionally, instructions for conducting an assay according to claim 1.


SEQUENCE LISTING (4 sequences)

SEQ ID NO: 1 - DNA, 28 bp, Artificial
  Description of artificial sequencePrimer
atggttttgtttaatygtagagttgttt

SEQ ID NO: 2 - DNA, 27 bp, Artificial
  Description of artificial sequencePrimer
taaacccraaaaaaaaaaacccaatat

SEQ ID NO: 3 - DNA, 24 bp, Artificial
  Description of artificial sequencePrimer
tttatttttatataagttttgttt

SEQ ID NO: 4 - DNA, 21 bp, Artificial
  Description of artificial sequencePrimer
cccttccctacaaaactatac